(19) United States
(12) Patent Application Publication
(10) Pub. No.: US 2023/0241082 A1
(43) Pub. Date: Aug. 3, 2023

(54) PULMONARY FORMULATION COMPRISING CANNABINOIDS

(71) Applicant: CANNOVEX BV, Diepenbeek (BE)

(72) Inventors: Bram BECKERS, Diepenbeek (BE); Steven PETERS, Diepenbeek (BE)

(21) Appl. No.: 18/002,189

(22) PCT Filed: Jun. 18, 2021
(86) PCT No.: PCT/EP2021/066641

§ 371 Date: Dec. 16, 2022

(30) Foreign Application Priority Data
Jun. 19, 2020 (EP) 20181174.2
Jun. 19, 2020 (EP) 20181177.5

(51) Int. Cl. A61K 31/00; A61K 9/00; A61K 9/16

(52) U.S. Cl. CPC A61K 31/658; A61K 9/0075; A61K 9/1623; A61K 9/1652

(57) ABSTRACT

The present invention relates to specific types of pharmaceutical formulations and compositions comprising cannabinoids for use in the treatment of various disorders.

### FIELD OF THE INVENTION

The present invention relates to specific types of pharmaceutical formulations and compositions comprising cannabinoids for use in the treatment of various disorders.

### BACKGROUND TO THE INVENTION

Research has shown various application domains for the use of cannabinoids over the past years. Therefore, the production of cannabinoid pharmaceuticals is gaining more and more attention. Although there is substantial evidence available for the therapeutic effects of cannabinoids, more research and clinical trials are of utmost importance in order to meet the demand of safe and efficient pharmaceutical formulations containing cannabinoids. More specifically in the domain of neurodegenerative diseases, the antioxidative, antiglutamatergic and anti-inflammatory are only some of the characteristics of cannabinoids which make them potential candidates in developing novel therapeutic strategies. Furthermore, in the domain of post-traumatic stress syndrome (PTSS), cannabinoids may form potential candidates in developing novel therapeutic strategies because of their role in the modulation of fear memory. There is some evidence that suggests that cannabinoids are beneficial for a range of other conditions, including blistering, blistering associated pain, as well as acute and chronic pain, such as neuropathic pain and headaches.

The choice of suitable pharmaceutical formulations greatly depends on factors such as: drug absorption (rate), metabolization and bioavailability. Therefore, a pharmaceutical formulation should be chosen bearing in mind the properties of the active substances as well as the desired drug release. For example, when a rapid uptake of active substances is required, formulations for pulmonary delivery may be suitable candidates. These pharmaceutical formulations provide for a fast absorption of the active substances into the systemic circulation due to the large surface area of the alveolar region, the thin air-blood barrier and the avoidance of the first pass-effect, altogether increasing the overall bioavailability of the active substances. A formulation for pulmonary delivery is especially useful for increasing the bioavailability of those active substances which are deactivated considerably by the liver (e.g. a number of cannabinoids). Formulations for pulmonary delivery have proven to be a valuable alternative to conventional oral drug therapy, such as capsules or tablets. Specific dosage forms such as liquid formulations, suspensions, powder (including micro- and nanometer-sized particles) or aerosols can be used to provide for the uptake of active substances via the lungs (e.g. delivery via inhalation).

Formulations for pulmonary delivery are particularly suitable for use in the treatment of neurodegenerative disorders, such as Alzheimer's disease (AD) and other dementias, Parkinson's disease (PD) and PD-related disorders, Essential Tremor, Multiple System Atrophy, Huntington's disease or Motor Neuron Diseases (MND). For different types of these neurodegenerative disorders, currently no cure exists. This is one of the reasons why there is a high need for treatments which could at least provide for symptom improvement, pain relief and/or increase of mobility, ultimately increasing the overall quality of life.

In the case of PTSS, there is a high need for treatments which could at least provide for symptom improvement, ultimately improving the overall quality of life of subjects suffering thereof. A number of symptoms may be tackled in order to effectuate such improvements: such as flashbacks, nightmares, avoidance, changes in physical and emotional reactions, uncontrollable thoughts about past events and severe anxiety.

The present invention fulfils the need of a cannabinoid pharmaceutical formulation with a high bioavailability, by providing for a novel formulation for pulmonary delivery comprising one or more cannabinoids being suitable for use in the treatment of disorders such as neurodegenerative disorders, blisters, post-traumatic stress syndrome and pain and the secondary symptoms caused by these disorders.

### SUMMARY OF THE INVENTION

The present invention relates to a combination comprising Tetrahydrocannabinol (THC) and Cannabidiol (CBD) for use in the treatment of a disorder selected from the list comprising: pain, neurodegenerative disorders, blisters, or post-traumatic stress syndrome, characterized in that said combination is formulated in a formulation for pulmonary delivery; in particular comprising at least one excipient, more in particular a saccharide.

In a following embodiment, the formulation as defined herein is selected from the list comprising: a liquid formulation, a suspension, a powder or an aerosol.

In another embodiment, the present invention provides a combination wherein the concentration of the excipient is about 5 to 10% (w/w).

In yet another embodiment, the present invention provides a combination wherein said excipient has a CBD/excipient, THC/excipient or CBD-THC/excipient ratio of about 1/1 to about 1/20 (w/w), preferably about 1/12 (w/w).

In a next embodiment, the formulation as defined herein is powder formulation comprising particles with an average particle diameter of about and between 0.1 to 100 μm, preferably about and between 0.1 and 10 μm, more preferably about and between 0.1 and 5 μm.

In yet another embodiment, the present invention provides powder formulation comprising particles produced with electrospraying.

In yet another embodiment, the aerosol as defined herein is a propellant-free aerosol.

In a following embodiment, the formulation as defined herein is formulated for administration by inhalation.

In a next embodiment, the formulation as defined herein is administered to a subject by means of an inhalation device.

In a further embodiments, said pain is selected from the list comprising: headaches, migraine, physiological pain, or physical ailments.

In a next embodiment, the present invention discloses a combination as defined herein, for use in the treatment of a disorder selected from the list comprising: pain, neurodegenerative disorders, post-traumatic stress syndrome or blisters, wherein said skin blisters are caused by burns or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

In a further embodiment, said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

In a next embodiment, the present invention provides a combination as defined herein for use in the treatment of a disorder selected from the list comprising: pain, neurodegenerative disorders, post-traumatic stress syndrome, or blisters is disclosed, wherein said use includes the alleviation of secondary symptoms caused by said disorders, such as selected from the list comprising (secondary) pain, itch, secondary impetigos, swelling, inflammation or bacterial infection.

In another embodiment, the present invention relates to a combination comprising Tetrahydrocannabinol (THC) and Cannabidiol (CBD) for use in the treatment of a disorder selected from the list comprising: pain, neurodegenerative disorders, blisters, or post-traumatic stress syndrome, characterized in that said combination is formulated in a formulation for pulmonary delivery, wherein said use includes the reduction of opioid consumption/dependency in the treatment of said disorders.

In another embodiment, the combination for use as defined herein may comprise one or more additional pharmaceutically active agents suitable for use in the treatment of said disorders.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Modulated differential scanning calorimetry of a formulation comprising CBD.

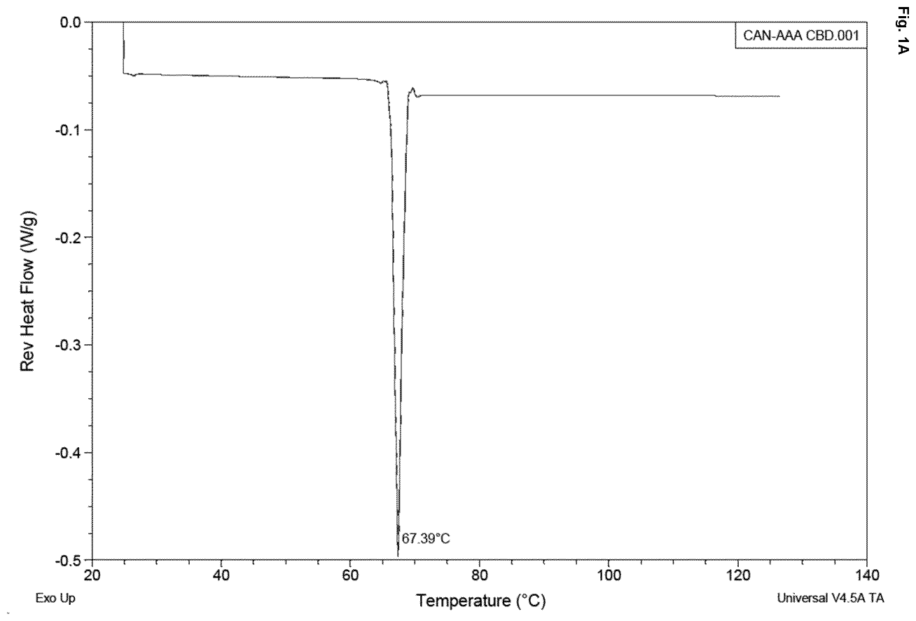

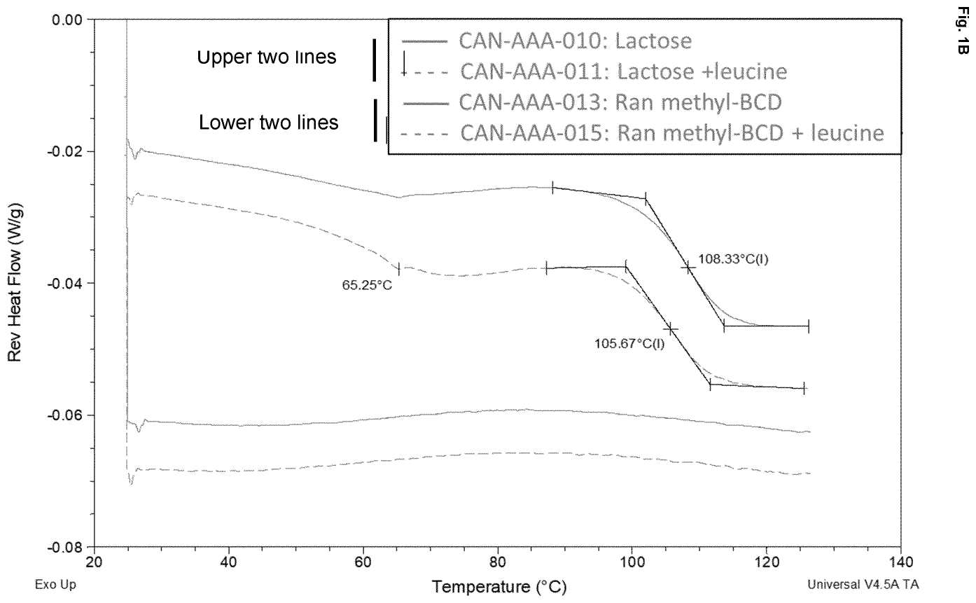

Pure CBD and a selection of SD samples were analyzed via modulated differential scanning calorimetry (mDSC) to evaluate the solid state of CBD in the spray dry powder. No melting peak of CBD (i.e. 67° C., FIG. 1A) was present on the mDSC graphs of the formulations with lactose or random methyl-BCD as excipients (i.e. without leucine) (FIG. 1B).

### DETAILED DESCRIPTION OF THE INVENTION

The present invention will be described with respect to particular embodiments and with reference to certain drawings, but the invention is not limited thereto. The drawings, as further described, are only schematic and non-limiting.

Furthermore, the terms first, second, further and the like in the description and in the claims are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a product comprising A and B” should not be limited to products consisting only of elements A and B. It means that, with respect to the present invention, the relevant elements of the product are A and B and that further components such as C may be present.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, FIGURE, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

As already detailed herein before, in a first aspect, the present invention provides a combination comprising Tetrahydrocannabinol (THC) and Cannabidiol (CBD) for use in the treatment of a disorder selected from the list comprising: pain, neurodegenerative disorders, post-traumatic stress syndrome, or blisters; characterized in that said combination is formulated in a formulation for pulmonary delivery.

In yet a further specific embodiment, the present invention provides a combination comprising Tetrahydrocannabinol (THC) and Cannabidiol (CBD) for use in the treatment of a disorder selected from the list comprising: pain, neurodegenerative disorders, post-traumatic stress syndrome, or blisters; characterized in that said combination is formulated in a formulation for pulmonary delivery.

Alternatively, the present invention relates to a combination of one or more cannabinoids or derivatives thereof for use in the treatment of a disorder selected from the list comprising: pain, neurodegenerative disorders, post-traumatic stress syndrome, or blisters, characterized in that said combination is formulated in a formulation for pulmonary delivery

In some embodiments, the present invention relates to a formulation for pulmonary delivery comprising THC.

In some embodiments, the present invention relates to a formulation for pulmonary delivery comprising CBD.

In some embodiments, the combination of CBD and THC may have a synergistic effect (e.g. in terms of anti-inflammatory activity) on the treatment of at least one of the disorders.

In some embodiments, CBD may decrease at least one of the potential side-effects caused by THC and vice versa. CBD may for example antagonize side-effects such as tachycardia and sedation caused by THC.

In some embodiments, at least one of the cannabinoids of the combination may have an immunoregulatory effect or immunomodulatory effect or may otherwise influence the immune system in order to support the treatment of one of said disorders.

As used herein and unless otherwise specified, the term “cannabinoid” is to be understood as a compound effectuating an activity involving the endocannabinoid system.

As used herein and unless otherwise specified, the term “endocannabinoid system” is to be understood as a cell-signaling system composed of endocannabinoids, being for example endogenous ligands of cannabinoid receptors (CB₁ and CB₂), and cannabinoid receptor proteins being expressed at the height of the vertebrate central nervous and peripheral nervous system.

In some embodiments, the cannabinoids including THC and CBD may be synthetically produced and/or plant-derived.

In some embodiments, the cannabinoids may be derived from Cannabis plants belonging to the Cannabis sativa L. species. Subspecies thereof may include: Cannabis sativa ssp. Sativa and ssp. Indica.

In some embodiments, the combination may comprise at least one Cannabis plant metabolite, including in particular: cannabinoids, terpenes, terpenoids, triglycerides, sterols, alkanes, squalenes, tocopherols, alkaloids or carotenoids.

As used herein and unless otherwise specified, the term “cannabis plants” is to be understood as a genus of flowering plants in the family of the Cannabaceae. Three main species can be recognized: Cannabis sativa, Cannabis indica and Cannabis ruderalis.

In some embodiments, the combination comprising THC and CBD may comprise THC and CBD in a ratio of THC:CBD from about 1:1000, 1:500, 1:250 to about 1000:1, 500:1, 250:1, preferably from about 1:100, 1:50, 1:25 to about 100:1, 50:1, 25:1 and more preferably from about 1:10 to about 10:1.

In some embodiments, the combination comprising THC and CBD may comprise THC and CBD in a ratio of THC:CBD from about 2:1 to about 1:2 In some embodiments, the combination comprising THC and CBD may comprise THC and CBD in a ratio of THC:CBD from about 1:1 to about 1:1

In some embodiments, the combination comprising THC and CBD may comprise an amount of THC of about 0.0625, 0.125, 0.25; 0.50; 0.75; 1; 2; 3; 4; 5; 10; 20; 30; 40; 50; 60; 70; 80; 90; 100 mg THC up to about 200; 300; 400; 500; 600; 700; 800; 900; 1000 mg THC and preferably an amount of THC of about 0.0625 to 100 mg THC.

In some embodiments, the combination comprising THC and CBD may comprise an amount of CBD of about 0.0625; 0.125, 0.25; 0.50, 0.75; 1, 2, 3, 4, 5, 10, 20, 30, 40, 50, 60, 70, 80, 90, 100 mg CBD up to about 200, 300, 400, 500, 600, 700, 800, 900, 1000 mg CBD.

In some embodiments, the combination may comprise at least one of the list comprising: THC CBD or other cannabinoids.

In some embodiments, these “other cannabinoids” may be selected from the list comprising the following types of cannabinoids: delta-9-trans-tetrahydrocannabinol (Δ⁹-THC) type, delta trans-tetrahydrocannabinol (Δ⁸-THC) type, cannabigerol (CBG) type, cannabichromene (CBC) type, cannabidiol (CBD) type, cannabinodiol (CBND) type, cannabielsoin (CBE) type, cannabicyclol (CBL) type, cannabinol (CBN) type, cannabitriol (CBT) type or miscellaneous-type cannabinoids.

The delta-9-trans-tetrahydrocannabinol (Δ⁹-THC) type includes molecules from the list comprising: Delta-9-tetrahydrocannabinol (THC), Delta-9-tetrahydrocannabinolic acid A (THCA-A), Delta-9-tetrahydrocannabinolic acid B (THCA-B), Delta-9-tetrahydrocannabivarin (THCV), Delta-9-tetrahydrocannabivarinic acid (THCVA), Delta-9-tetrahydrocannabiorcol (THCO), Delta-9-trans-tetrahydrocannabiorcolic acid (THCOA), Delta-9-tetrahydrocannabinol-C4 (THC-C4), Delta-9-trans-tetrahydrocannabinolic acid-C4 (THCA-C4), β-fenchyl-Delta-9-tetrahydrocannabinolate, α-fenchyl-Delta-9-tetrahydrocannabinolate, epi-bornyl-Delta-9-tetrahydrocannabinolate, bornyl-Delta-9-tetrahydrocannabinolate, α-terpenyl-Delta-9-tetrahydrocannabinolate, 4-terpenyl-Delta-9-tetrahydrocannabinolate, α-cadinyl-Delta-9-tetrahydrocannabinolate, γ-eudesmyl-Delta-9-tetrahydrocannabinolate or Cannabisol.

The delta-8-trans-tetrahydrocannabinol (Δ⁸-THC) type includes molecules from the list comprising: Delta-8-trans-tetrahydrocannabinol (Δ⁸-THC) or Delta-8-trans-tetrahydrocannabinolic acid (Δ⁸-THCA).

The cannabigerol (CBG) type includes molecules from the list comprising: Cannabigerol (CBG), Cannabigerolic acid (CBGA), Cannabigerol monomethyl ether (CBGM), Cannabigerolic acid monomethyl ether (CBGAM), Cannabigevarin (CBGV), Cannabigerovarinic acid (CBGVA), Cannabinerolic acid ((Z)-CBGA), γ-eudesmyl-Cannabigerolate, α-cadinyl-Cannabigerolate, 5-acetyl-4-hydroxycannabigerol, 4-acetoxy-2-geranyl-5-hydroxy-3-n-pentylphenol, (±)-6,7-trans-epoxycannabigerolic acid, (±)-6,7-cis-epoxycannabigerolic acid, (±)-6,7-cis-epoxycannabigerol, (±)-6,7-trans-epoxycannabigerol, Carmagerol-dihydroxy-CBG or Sesquicannabigerol.

The cannabichromene (CBC) type includes molecules from the list comprising: Cannabichromene (CBC), Cannabichromenic acid (CBCA), Cannabichromevarin (CBCV), Cannabichromevarinic acid (CBCVA), Cannabichromene C3 (CBC-C3), (±)-4-acetoxycannabichromene, (±)-3″-hydroxy-Δ4″-cannabichromene or (−)-7-hydroxycannabichromane.

The cannabidiol (CBD) type includes molecules from the list comprising: Cannabidiol (CBD), Cannabidiolic acid (CBDA), Cannabidivarin (CBDV), Cannabidivarinic acid (CBDVA), Cannabidiol monomethyl ether (CBDM), Cannabidiorcol (CBD-C1), Cannabidiol-C4 (CBD-C4) or Cannabimovone.

The cannabinodiol (CBND) type includes molecules from the list comprising: Cannabinodiol (CBND-05) or Cannabinodivarin (CBND-C3).

The cannabielsoin (CBE) type includes molecules from the list comprising: Cannabielsoin (CBE), Cannabielsoic acid A (CBEA-A), Cannabielsoic acid B (CBEA-B), Cannabielsoin-C3 (CBE-C3), Cannabielsoic-C3 acid B (CBEA-C3 B), Cannabiglendol-C3-OH-iso-HHCV-C3, Dehydrocannabifuran (DCBF) or Cannabifuran (CBF).

The cannabicyclol (CBL) type includes molecules from the list comprising: Cannabicyclol (CBL), Cannabicyclolic acid (CBLA) or Cannabicyclovarin-CBLV (CBL-C3).

The cannabinol (CBN) type includes molecules from the list comprising: Cannabinol (CBN), Cannabinolic acid (CBNA), Cannabivarin-CBV (CBN-C3), Cannabinol-C4 (CBN-C4), Cannabinol-C2 (CBN-C2), Cannabiorcol (CBN-C1), Cannabinol methyl ether (CBNM), 4-terpenyl-Cannabinolate, 8-Hydroxycannabidiol (8-OH-CBN) or 8-Hydroxycannabidiolic acid (8-OH-CBNA).

The cannabitriol (CBT) type includes molecules from the list comprising: (−)-trans-Cannabitriol ((−)-trans-CBT-C5), (+)-trans-Cannabitriol ((+)-trans-CBT-C5), cis-Cannabitriol ((±)-CBT-C5), (−)-trans-10-ethoxy-9-hydroxy-Δ6a(10a)-tetrahydrocannabinol ((−)-trans-CBT-OEt-05), trans-Cannabitriol-C3 ((±)-trans-CBT-C3), CBT-C3-homoloog, trans-10-ethoxy-9-hydroxy-Δ6a(10a)-tetrahydrocannabivarin-C3 ((−)-trans-CBT-OEt-C3), 8,9-dihydroxy-Δ6a(10a)-tetrahydrocannabinol (8,9-Di-OH-CBT-C5), Cannabidiolic acid A cannabitriol ester (CBDA-C5 9-OH-CBT-C5 ester), Cannabitriolvarin (CBTV) or ethoxy-Cannabitriolvarin (CBTVE).

The miscellaneous-type cannabinoids includes molecules from the list comprising: Cannabifuran (CBF), Dehydrocannabifuran (DCBF), Cannabitetrol (CBTT), Cannabiripsol (CBR), Cannabicitran (CBR-C3), Cannabioxepane (CBX), Cannabicoumaronone (CBCON), Cannabicoumaronic acid, Cannabiglendol-C3 (OH-iso-HHCV-C3), 10-Oxo-Δ6a(10a)-tetrahydrocannabinol (OTHC), (−)-Δ9-cis-(6aS,10aR)-tetrahydrocannabinol (cis-Δ9-THC), 4-acetoxy-2-geranyl-5-hydroxy-3-n-pentylphenol, 2-geranyl-5-hydroxy-3-n-pentyl-1,4-benzoquinone, 5-acetoxy-6-geranyl-3-n-pentyl-1,4-benzoquinone, 8α-hydroxy-Δ9-tetrahydrocannabinol, 8β-hydroxy-Δ9-tetrahydrocannabinol, 10α-hydroxy-Δ8-tetrahydrocannabinol, 10β-hydroxy-Δ8-tetrahydrocannabinol, 10α-hydroxy-Δ9,11-hexahydrocannabinol, 9β,10β-epoxoexahydrocannabinol or 11-acetoxy-Δ9-tetrahydrocannabinolic acid A.

The use of a formulation for pulmonary delivery may increase the bioavailability of at least one of the active substances compared to other formulations. For example, when using oral drug delivery, the drugs are subject to hepatic drug metabolization (hereinafter also called: first pass effect) before reaching the systemic circulation. Cannabinoids in particular are substantially deactivated when subject to this first pass effect. The use of formulations avoiding this first pass effect, such as formulations for pulmonary delivery, therefore increases the overall bioavailability of cannabinoids, making it a suitable pharmaceutical formulation. When compared to oral delivery, inhalation of said formulations provides for a more rapid onset of pharmacological action and peak plasma levels.

Formulations for pulmonary delivery accommodate an uptake of active substances via the lungs. This pulmonary route of drug delivery encompasses in particular the bronchi, bronchioles and alveoli being the main absorption zone and via which this first pass effect is bypassed. This is an advantage compared to e.g. oral drug delivery. Furthermore, the pulmonary route may provide for active or passive transport of molecules (e.g. active substances) through the alveolar epithelium, the capillary epithelium and the lymph-containing interstitial space between these two cellular layers. Generally speaking, the smaller the molecules, the faster this transport occurs.

In some embodiments, at least 0.1, 0.2, 0.5, 1, 2, 5, 10, 20, 30, 40, 50, 60, 70, 80, 90, 95% of the active substances of the formulations for pulmonary delivery may reach the pulmonary region and more specifically the alveolar region.

In some embodiments, the formulation for pulmonary delivery may be an aqueous or a non-aqueous formulation.

In some embodiments, the formulation for pulmonary delivery may comprise at least one propellant. Examples of such propellants may be selected from the list comprising: fluorochloro hydrocarbons, compressed gas, propane, n-butane, isobutane, dimethyl ether, methyl ethyl ether, nitrous oxide, hydrofluoroalkanes (HFA) and carbon dioxide.

In some embodiments, the formulation for pulmonary delivery may comprise a solvent such as selected from the list comprising: inorganic solvent and organic solvent.

The amount of solvent may have an impact on the droplet size comprised within at least some of the formulations for pulmonary delivery.

The solvents include molecules from the list comprising: ethanol, propanol, propylene glycol, glycerol, polyethylene glycol and sub-micron liposomal dispersion (microemulsions and micellar solutions). In some embodiments, at least a part of the cannabinoids within the formulation for pulmonary delivery may be dissolved within said solvent.

In some embodiments, the formulation for pulmonary delivery may comprise a oligo- or polysaccharide such as selected from the list comprising: water soluble complex carbohydrates, such as a starch, a polyol or sugar alcohol, maltodextrin, (2-Hydroxypropyl)-beta-cyclodextrin (HPBCD), or random methyl-beta-cyclodextrin (RMBCD), cellulose or cellulose derivatives such as hydroxypropyl methylcellulose (HPMC), hydroxyethyl methyl cellulose (NEMC), hydroxypropyl methylcellulose acetate succinate (HPMCAS), sodium or calcium alginate, acacia gum, xantham gum, guar gum, or combinations thereof.

In some embodiments, the polysaccharide may comprise cellulose or cellulose derivatives.

In some embodiments, the formulation for pulmonary delivery may comprise an emulsifier such as e.g. selected from the list comprising: lecithin, Acconon mixtures, Capmul MCG, propylene glycol esters, Caprol polyglycerol esters, Captex medium chain esters, Kolliphor EL, Kolliphor RH40, Poloxamers, polysorbates and Tween 80.

In some embodiments, the formulation for pulmonary delivery may comprise a mono- or disaccharide such as selected from the list comprising: glucose, dextrose, fructose, sucrose, lactose, trehalose, mannitol, maltose or isomaltose.

In some embodiments, delivery devices may be used to deliver the formulations for pulmonary delivery to the subject. Such delivery devices may include nebulizers, metered-dose inhalers (MDIs), dry powder inhalers (DPIs) and soft mist inhalers (SMI).

As used herein and unless otherwise specified, the term “nebulizers” is to be understood as a drug delivery device which is used to administer active substances into the lungs in the form of a mist. Generally, oxygen, compressed air or ultrasonic power is used for transforming liquid solutions or suspensions into aerosol droplets which can be inhaled directly. In some embodiments, said liquid solutions or suspensions may be sprayed under high pressure through small nozzles so as to form inhalable aerosol droplets using said nebulizers.

Propellant-free nebulizers are a type of nebulizers having the main advantage of eliminating the use of propellant gases such as for example fluorochloro hydrocarbons.

In some embodiments, said inhalable aerosol droplets may have an average droplet size of less than 100, 90, 80, 70, 60, 50, 40, 30, 20, 15, 10, 9, 8, 7, 6, 5, 4, 3, 2, 1, 0.5, 0.1 μm.

As used herein and unless otherwise specified, the term “metered-dose inhaler (MDI)” is to be understood as a drug delivery device which is used to administer active substances into the lungs in the form of an aerosol, wherein a propellant (e.g. hydrofluorocarbons) and optionally at least one stabilizing excipients are added to the formulation in order to accommodate the drug delivery.

As used herein and unless otherwise specified, the term “dry powder inhaler (DPI)” is to be understood as a drug delivery device which is used to administer active substances into the lungs in the form of a dry powder, said powder generally comprising micrometer- or nanometer-sized particles and optionally at least one stabilizing excipient to accommodate the drug delivery. Generally, subject inhalation is necessary in order for the drug to enter the lungs. Examples disclosed herein (example 1) show how a formulation for a dry powder inhaler may be composed.

As used herein and unless otherwise specified, the term “soft mist inhaler (SMI)” is to be understood as a drug delivery device which is used to administer active substances into the lungs in the form of a fine mist (aerosol). Generally, subject inhalation is necessary in order for the drug to enter the lungs. Also, generally a propellant-free aerosol is used in SMI devices. Examples disclosed herein (example 2) show how a formulation for a soft mist inhaler may be composed.

In some embodiments, at least one of the cannabinoids of the formulation for pulmonary delivery may cause bronchodilatation. More specifically when said formulations for pulmonary delivery is delivered to the subject by means of inhalation (e.g. by means of delivery devices).

In some embodiments, the combination may be administered multiple times a day and more specifically at least once, twice, three times, four times, five times, six times, seven times, eight times, nine times, ten times a day.

In another embodiment, the combination may be administered not more than three, four, five, six, seven, eight, nine, ten times a day.

In some embodiments, the combination may be administered by the subject. Formulations for pulmonary delivery are generally suitable to be administered by the subject without any additional assistance. However, if the condition of the subject does not permit self-administration, said formulations may be administered by others (e.g. nurses).

In some embodiments, the combination may be required to be diluted. The dilution may for example be performed using a certain amount of physiologic serum (e.g. 0.9% NaCl solution).

In some embodiments, at least one of the substances within the formulations for pulmonary delivery is at least partially systemically absorbed.

In some embodiments, at least one of the substances within the formulations for pulmonary delivery may have local effects when inhaled. These local effects may comprise effects on the bronchi (e.g. bronchodilation).

In some embodiments, the formulation for pulmonary delivery may comprise a minimum amount of THC of at least about 0.01, 0.02, 0.03, 0.04, 0.05, 0.1, 0, 0.5, 1, 2, 3, 4, 5, 10, 15, 20, 25, 30, 40, 50 wt %.

In some embodiments, the formulation for pulmonary delivery may comprise a minimum amount of CBD of at least about 0.01, 0.02, 0.03, 0.04, 0.05, 0.1, 0.2, 0.5, 1, 2, 3, 4, 5, 10, 15, 20, 25, 30, 40, 50 wt %.

In some embodiments, the formulation for pulmonary delivery may comprise a maximum amount of THC of up to about 60, 70, 80, 90, 95 wt %.

In some embodiments, the formulation for pulmonary delivery may comprise a maximum amount of CBD of up to about 60, 70, 80, 90, 95 wt %.

As used herein and unless otherwise specified, the term “wt %” is also referred to as weight percentage and is to be understood as the mass of a component divided by the total mass of the mixture and then multiplied by 100.

In a following embodiment, the formulation as defined herein is selected from the list comprising: a liquid formulation, a suspension, a powder or an aerosol.

In some embodiments, said powder formulations may comprise micrometer- or nanometer-sized particles.

In some embodiments, the formulation as defined herein may be formulated in a semi-solid form or crystallized form.

As used herein and unless otherwise specified, the term “liquid formulation” is to be understood as all types of formulations which comprise a liquid component and at least one active substance. Examples include for example the at least one active substance being dissolved or suspended within said liquid.

As used herein and unless otherwise specified, the term “suspension” is to be understood as all types of formulations being heterogeneous systems comprising two phases and more specifically which comprise a first substance (i.e. the dispersed phase) being uniformly distributed throughout a second substance (i.e. the continuous phase) and being insoluble in said continuous phase. Generally, it concerns a fluid continuous phase and a solid dispersed phase.

As used herein and unless otherwise specified, the term “powder” is to be understood as a dry, bulk solid composed of multiple very fine particles having the ability to flow.

As used herein and unless otherwise specified, the term “aerosol” is to be understood as a suspension system of particles (i.e. solid or liquid) in a gas (e.g. air).

In yet another embodiment, the aerosol as defined herein is a propellant-free aerosol.

In a following embodiment, the formulation as defined herein is formulated for administration by inhalation. Formulations which could be inhaled can be for example selected from the list comprising: powders, vapors, suspensions and aerosols.

In some embodiments, said powders for inhalation may comprise micrometer- or nanometer-sized particles.

For the avoidance of doubt, the components, concentrations, ratio's, . . . defined herein below for “a formulation” may equally apply for a dry powder formulation, a liquid formulation, a suspension, or an aerosol formulation as well as for the starting material (or liquid feed) to produce such formulation.

As used herein, the term “liquid feed” is to be understood as starting material or starting liquid to be converted to a liquid formulation, a suspension, a powder or an aerosol, comprising an active pharmaceutical ingredient (API) such as CBD, THC or CBD/THC and optionally one or more other excipients.

In addition, the concentration of a component in the liquid feed may not necessarily be the same as the final concentration of the formulation of the present invention.

As used herein and unless otherwise specified, the term “excipient” is to be understood as a substance formulated alongside the API, which may be used for long-term stabilization, bulking up formulations that contain potent active ingredients in small amounts (thus often also referred to as “bulking agents”, “fillers”, or “diluents”), or to confer a therapeutic enhancement on the active ingredient in the final dosage form, such as facilitating drug absorption, reducing viscosity or enhancing solubility. Excipients may also be used in the manufacturing process, to aid in the handling of the active substance concerns such as by facilitating powder flowability non-stick properties, in addition to aiding in vitro stability such as prevention of denaturation or aggregation over the expected shelf life.

Further, as used herein, an excipient may be any substrate used in the process of drug delivery which serves to improve the selectivity, effectiveness, and/or safety of drug administration. Different methods of attaching the drug to an excipient have been implemented, including adsorption, integration into the bulk structure, encapsulation, and covalent bonding.

In a further embodiment, the combination of the present invention may further comprise one or more excipients. In the context of the present invention, excipients may include nanoemulsions, dendrimers, micelles, liposomes, solid lipid nanoparticles and nanoparticles of biodegradable polymers.

In some embodiments, said formulations for pulmonary delivery may further comprise excipients such as a lipid carrier, a polymeric carrier, microsphere carrier, preferably a polymeric carrier.

In a preferred embodiment, said formulations for pulmonary delivery may comprise at least one excipient being a saccharide.

In another embodiment, said formulations for pulmonary delivery may further comprise at least one an excipient selected from the list comprising: ethanol, sugars, including saccharides and polysaccharides, such as lactose, glucose, dextrose, fructose, mannose, sucrose, mannitol, trehalose, maltose or isomaltose; water soluble complex carbohydrates, such as a starch, a polyol or sugar alcohol, maltodextrin, (2-Hydroxypropyl)-beta-cyclodextrin (HPBCD), or random methyl-beta-cyclodextrin (RMBCD), cellulose or cellulose derivatives such as hydroxypropyl methylcellulose (HPMC), hydroxyethyl methyl cellulose (NEMC), hydroxypropyl methylcellulose acetate succinate (HPMCAS), sodium or calcium alginate, acacia gum, xantham gum, guar gum; citrates, amino acids such as glycine, L-leucine, isoleucine, trileucine, tartrates, methionine, vitamin A, vitamin E, zinc citrate, trisodium citrate, zinc chloride, polyvinylpyrrolidone, polysorbate 80, phospholipids including diphosphotidylcholine; surfactants such as lecithin, and the like.

In some embodiments, the polysaccharide may comprise cellulose or cellulose derivatives.

In some embodiments, the formulation for pulmonary delivery may comprise at least one excipient at a concentration of about 0.25, 0.5, 0.75, 1, 2, 3, 4, 5, 6, 7, 8, 9, 10, 11, 12, 13, 14, 15, 16, 17, 18, 19, 20%.

For example, examples 3 (CBD formulation) and 4 (THC and CBD/THC formulation) provide a method wherein lactose or RMBCD are used as excipients to manufacture a dry powder for inhalation. These methods are merely indicative to components and concentrations and thus examples are not limited to those specific components and concentrations used.

In some embodiments, said formulation for inhalation may comprise an active pharmaceutical ingredient (API) (such as CBD, THC or CBD/THC) and an excipient in a API/excipient ratio of about 1/1, 1/2, 1/3, 1/4, 1/5, 1/6, 1/7, 1/8, 1/9, 1/10, 1/11, 1/13, 1/14, 1/15, 1/16, 1/17, 1/18; 1/19; 1/20 (w/w) to about 2/1, 3/1, 4/1, 5/1, 6/1, 7/1, 8/1, 9/1, 10/1, 11/1, 12/1, 13/1, 14/1, 15/1, 16/1, 17/1, 18/1, 19/1, 20/1 (w/w), and preferably about 1/12 (w/w).

For example in example 3, a specific liquid feed is provided, which comprises CBD and lactose in a 1/12 (w/w) ratio meaning CBD has a concentration of about 7.3% and lactose of about 87.9% in the liquid feed. The final concentration of CBD and lactose in the formulation might be equal or less than the initial feed concentration.

For some excipients, increasing the amount of excipient from for example about 1/12 (w/w) to about 1/15 (w/w) might lead to reduced stickiness of the powder, better encapsulation of the API and therefore a better particle yield.

In a preferred embodiment, said formulations for pulmonary delivery may comprise at least one saccharide.

In some embodiments, the formulation for pulmonary delivery may comprise at least one saccharide at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise mannitol at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise maltodextrin at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise trehalose at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise lactose at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise (2-Hydroxypropyl)-beta-cyclodextrin (HPBCD) at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise random methyl-beta-cyclodextrin (RMBCD) at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise leucine at a concentration of about 0.25, 0.5, 0.75, 1, 2, 3, 4, 5, 6, 7, 8, 9, 10, 11, 12, 13, 14, 15, 16, 17, 18, 19, 20%.

In some embodiments, the formulation for pulmonary delivery may comprise lecithin at a concentration of about 0.25, 0.5, 0.75, 1, 2, 3, 4, 5, 6, 7, 8, 9, 10, 11, 12, 13, 14, 15, 16, 17, 18, 19, 20%.

In a preferred embodiment, said formulations for pulmonary delivery may comprise lactose and leucine.

In another preferred embodiment, said formulations for pulmonary delivery may comprise about 7% (w/w) lactose and about 5% (w/w) leucine.

In another preferred embodiment, said formulations for pulmonary delivery may comprise about 7% (w/w) lactose and about 10% (w/w) leucine.

In yet another preferred embodiment, said formulations for pulmonary delivery may comprise about 7% (w/w) RMBCD.

In some embodiments, said formulation for inhalation is obtained by converting a liquid feed to said formulation using a biopharmaceutical conversion process, such as lyophilization or a liquid atomization technique, in particular electrospraying, spray drying or freeze-drying. Liquid atomization techniques are suitable for making drug-encapsulated nanoparticles and for the production of dry nanoparticle powders.

As used herein and unless otherwise provided, the term “electrospraying” is to be understood as an electro-hydrodynamic atomization (EHDA) method using an electrical field allowing for the breakdown of a conductive liquid jet, flowing through a capillary nozzle, into fine droplets with high monodispersity. Electrospraying is a versatile and inexpensive way to produce nanoparticles and nanosuspensions.

As used herein and unless otherwise provided, the term “spray drying” is to be understood as a process whereby a liquid feed is converted into a dry powder in a single step. The process is typically performed by first atomizing the solution into fine droplets that are then dried in a large chamber by using warm gas quickly to form nanostructured microparticles. The resulting dry particles are collected with a cyclone. Parameters of the process such as the liquid feed rate, atomization pressure, nozzle air rate, inlet air flow, outlet temperature, cyclone gas and/or spray rate are process conditions that may be adapted to tailor the particle size and morphology and thus retrieve a suitable formulation. Further details on these parameters may be found in the examples part. These nanostructured microparticles are easier to formulate into tablets, for example, and can be redispersed as nanoparticles in water.

Examples disclosed herein show a dry powder formulation derived with electrospraying (example 1) as well as dry powder formulations derived with spray drying (example 3 and 4).

In some embodiments, said formulation for inhalation may comprise particles produced with a liquid atomization technique, in particular electrospraying or spray drying.

In another embodiment, said formulation for inhalation may comprise particles produced with electrospraying. In a further embodiment, said formulation for inhalation may comprise particles produced with spray drying.

In some embodiments, said formulation for inhalation may comprise micrometer- or nanometer-sized particles.

As used herein, the term “micrometer-sized particles” (particles in the range of the μm scale) as well as the term “nanometer-sized particles” (particles in the range of the nm scale) refer to “inhalable particles” which can be aspirated into the nose or mouth. Accordingly, both terms can be used interchangeably in the context of inhalable particles. For example, micrometer-sized particles for pulmonary delivery having a diameter of between about 0.5 (i.e. 500 nm) and about 10 μm (10 000 nm) can reach the lungs and can reach the systemic circulation and deliver an active agent. A diameter of less than about 10 μm is desirable to navigate the turn of the throat and a diameter of about 0.5 μm or greater is desirable to avoid being exhaled. Generally, micrometer-sized particles having diameters greater than 10 μm or greater than 20 μm are useful for local delivery to the respiratory tract and lungs. Micrometer-sized particles having a diameter of between about 0.5 and about 10 microns can reach the lungs, successfully passing most of the natural barriers.

In some embodiments, said formulation for pulmonary delivery comprises micrometer- or nanometer-sized particles with an average particle diameter of less than about 100, 90, 80, 70, 60, 50, 40, 30, 20, 15, 10, 9, 8, 7, 6, 5, 4, 3, 2, 1; 0.5, 0.1 μm.

In a preferred embodiment, said formulation for pulmonary delivery comprises micrometer- or nanometer-sized particles with an average particle diameter of about and between 0.1 to 100 μm, preferably about and between 0.1 to 50 μm, more preferably about and between 0.1 to 10 μm, most preferably 0.1 to 5 μm. Examples (example 3 and 4) disclosed herein show a dry powder formulation with micrometer particles in a range of about 0 to 5 μm comprising an active ingredient such as CBD or THC.

In some embodiments, said formulation for pulmonary delivery may comprise wrinkled particles. As used herein, the term “wrinkled particle” are to be understood as a particle with wrinkled morphology, i.e. not having a smooth surface, which have significantly enlarged surface areas and enhances the aerodynamics of aerosol in dry powder inhalation. For example, wrinkled particles spray-dried in the presence of leucine may enhance the dispersibility, leading to high fine particle fraction. In particular and as exemplified in example 3, using a formulation comprising HPBCD or random methyl-BCD more wrinkled particles were obtained due to the smaller diffusion rate of these large molecules (i.e. cyclodextrins) and this might lead to a formulation with better inhalation properties.

In some embodiments, the morphology and structure of said formulation for pulmonary delivery is characterized using x-ray diffraction.

In a next embodiment, the formulation as defined herein is administered to a subject by means of an inhalation device.

As used herein and unless otherwise specified, the term “inhalation device” is to be understood as an apparatus accommodating the delivery of formulations for pulmonary delivery to the subject and more specifically to the respiratory system such as the lungs. Examples of inhalation devices include for example: nebulizers, metered dose inhalers, dry powder inhalers and soft mist inhalers.

In some embodiments, said inhalation device may provide for a dosage form selected from the list comprising: single dosage or multi dosage form.

In a next embodiment, the present invention discloses a combination as defined herein, for use in the treatment of a disorder selected from the list comprising: pain, neurodegenerative disorders, post-traumatic stress syndrome, or blisters, wherein said skin blisters are caused by burns or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

As used herein and unless otherwise specified, the term “blisters” is to be understood as a bulge of the upper layers of the skin, filled with body fluid such as serum or plasma and generally caused by for example infections, burning or friction, or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

As used herein and unless otherwise specified, the term “skin allergies” is to be understood as a reaction to an allergen or irritant, which may lead to symptoms such as itchiness, redness of the skin, rashes and blistering of the skin. Treatment includes treatment of the underlying conditions which cause the skin allergy and treatment of the symptoms.

As used herein and unless otherwise specified, the term “eczema” (may also be referred to as: dermatitis”) is to be understood as a group of diseases being only partly interrelated and resulting in inflammation of the skin and characterized by itchiness, red skin, rashes, skin thickening and small blisters.

In some embodiments, the different forms of eczema may be selected from the list comprising: atopic dermatitis, contact dermatitis, dyshidrotic eczema, nummular eczema, seborrheic dermatitis or stasis dermatitis.

As used herein and unless otherwise specified, the term “bullous pemphigoid” is to be understood as an autoimmune pruritic skin disease. It is a type of pemphigoid, which is a group of autoimmune blistering skin diseases. Bullous pemphigoid may by characterized by the formation of blisters within the (epi)dermal skin layers and the formation of anti-hemidesmosome antibodies.

As used herein and unless otherwise specified, the term “bullous impetigo” is to be understood as a bacterial skin infection resulting in large blisters, mainly in skin fold areas (e.g. groin, armpit). The blisters are caused by exfoliative toxins which are produced by Staphylococcus aureaus, causing the intercellular connections of the epidermis to fall apart.

As used herein and unless otherwise specified, the term “dermatitis herpetiformis” is to be understood as a chronic autoimmune skin condition characterized by fluid-filled blisters, chronic papulovesicular eruptions and intense itchiness. Dermatitis herpetiformis is a cutaneous manifestation of Coeliac disease and symptoms are chronic and are linked to the amount of gluten ingested.

As used herein and unless otherwise specified, the term “pemphigus vulgaris” is to be understood as a chronic skin disease characterized by blistering of the skin. It is a type II hypersensitivy reaction with antibody formation against desmosomes. The attack of these desmosomes by the antibodies causes the skin layers to be separated, which resembles blisters.

As used herein and unless otherwise specified, the term “mucous membrane pemphigoid” (may also be refered to as: MMP”) is to be understood as a group of chronic autoimmune subepithelial blistering diseases which primarily involves the mucous membranes and sometimes the skin.

As used herein and unless otherwise specified, the term “pemphigoid gestationis” is to be understood as a pregnancy-associated autoimmune skin disease. Often, an itchy rash develops into blisters. It may sometimes also be referred to as herpes gestationis.

As used herein and unless otherwise specified, the term “epidermolysis bullosa” is to be understood as a group of genetic skin conditions characterized in the formation of blisters of the mucous membranes and the skin. The conditions cannot be cured, although wound care and pain relief are often applied.

As used herein and unless otherwise specified, the term “pemphigus foliaceous” is to be understood as an autoimmune blistering disease of the skin. Skin lesions are often crusted erosions with an erythematous base.

As used herein and unless otherwise specified, the term “toxic epidermal necrolysis” is to be understood as a potentially life-threatening skin disorder, resulting in skin blistering and affected mucous membranes.

In some embodiments, the combination as defined herein, or at least one of the cannabinoids of the combination may alleviate (at least one of the symptoms of said disorders.

Examples of these blister-related symptoms comprise: skin or oral mucous itchiness, redness, rashes, thickening, hypersensitivity, bleeding, pain.

As used herein and unless otherwise specified, the term “neurodegenerative disorders” is to be understood as an umbrella term for a number of conditions primarily affecting brain neurons. These conditions are often incurable and debilitating and often result in progressive degeneration and/or death of nerve cells.

In a further embodiment, said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

As used herein and unless otherwise specified, the term “Alzheimer's disease (AD)” is to be understood as a chronic neurodegenerative disease. The disease is often characterized by among others: disorientations, mood swings and behavioral issues. Besides that, it is the most common cause of dementia.

As used herein and unless otherwise specified, the term “Parkinson's disease (PD)” is to be understood as a neurodegenerative disease of the central nervous system, affecting the motor system. Symptoms include rigidity, walking difficulties and shaking.

As used herein and unless otherwise specified, the term “PD-related disorders” is to be understood as a group of disorders related to PD itself and/or the pharmacological management of the disease, including dopamine deficiency syndrome, dopamine dependency syndrome, impulse control disorders and dopamine dysregulation syndrome.

As used herein and unless otherwise specified, the term “Essential Tremor” is to be understood as a progressive neurological disorder, which may affect all parts of the body. It causes involuntary, rhythmic contractions and relaxations of certain muscle groups. Furthermore, it is the most common movement disorder.

As used herein and unless otherwise specified, the term “Multiple System Atrophy” is to be understood as a progressive neurodegenerative disorder, caused by progressive degeneration of neurons in several parts of the brain (e.g. cerebellum and basal ganglia). It is characterized by among others slow movement, tremors and autonomic dysfunction.

As used herein and unless otherwise specified, the term “Huntington's disease” is to be understood as an inherited disease causing progressive degeneration of brain nerve cells, resulting in cognitive, physical and psychiatric disorders. Signs and symptoms tend to develop between the age of 30 to 40.

As used herein and unless otherwise specified, the term “Motor Neuron Diseases (MND)” is to be understood as a group of neurodegenerative diseases affecting motor neurons, causing moement-related symptoms (e.g. muscle weakness). The group includes: progressive bulbar palsy, amyotrophic lateral sclerosis, progressive muscular atrophy, monomelic amyotrophy and primary lateral sclerosis.

In some embodiments, at least one of the cannabinoids of the combination may delay or impede the progression of (at least one of the symptoms of) said disorders.

Examples of neurodegenerative disease-related symptoms comprise: memory impairment, difficulties with regard to coordination, (painful) muscle spasms, spasticity, mobility, tremor, rigidity, aphasia, speaking difficulties, aggressiveness, sleeping disorders, weakness, paralysis, appetite loss, depression, drooling, visual hallucinations, urinary dysfunction, glaucoma, bronchial asthma, emesis and agitation.

In some embodiments, at least one of the cannabinoids of the combination may improve functional recovery.

In some embodiments, at least one of the cannabinoids of the combination may reduce demyelination and/or induce remyelination.

In some embodiments, at least one of the cannabinoids of the combination may be used to achieve significantly better intracranial pressure/cerebral perfusion pressure control.

In some embodiments, at least one of the cannabinoids of the combination may have neuroprotective effects.

In some embodiments, at least one of the cannabinoids of the combination may decrease the secretion of inflammatory cytokines.

As used herein and unless otherwise specified, the term “post-traumatic stress syndrome (also referred to as: PTSS)” is to be understood as a mental health condition often triggered by the experience or witnessing of a terrifying event. PTSS symptoms include intrusive memories, negative changes of thoughts and mood, changes relating to physical and emotions reactions and avoidance.

In some embodiments, at least one of the cannabinoids of the combination may at least reduce or impede (at least one of the symptoms of) said disorders.

Examples of PTSS-related symptoms comprise: intrusive memories (e.g. recurrent, unwanted distressing memories of the traumatic event, flashbacks, upsetting dreams or nightmares about the traumatic event and severe emotional distress or physical reaction to elements reminding someone about the traumatic event), avoidance (e.g. avoiding thoughts or conversations linked to the traumatic event, avoiding activities, locations or people reminding someone about the traumatic event), negative changes in thinking and mood (e.g. negative thoughts about themselves or others, hopelessness with regard to the future, memory problems, difficulty of maintaining close relationships, a feeling of detachment from family and friends, a lack of interest in certain activities, difficulty of experiencing positive emotions and a feeling of emotional numbness) and changes in physical and emotion reactions (e.g. being easily startled/frightened, having a persisting urge of being on guard for danger, self-destructive behavior (e.g. drinking large amounts of alcohol, driving too fast), sleeping problems, concentration problems, irritability, anger outbursts, aggressiveness, overwhelming guilt or shame).

In some embodiments, the combination as defined herein, or at least one of the cannabinoids of the combination may reduce anxiety.

In some embodiments, the combination as defined herein, or at least one of the cannabinoids of the combination may reduce depression.

In some embodiments, the combination as defined herein, or at least one of the cannabinoids of the combination may contribute to the reduction of amygdala hyperactivation.

In some embodiments, the combination as defined herein, or at least one of the cannabinoids of the combination may have neuroprotective effects.

In some embodiments, the combination as defined herein, or at least one of the cannabinoids of the combination may decrease suicidality.

In some embodiments, the combination as defined herein, or at least one of the cannabinoids of the combination may decrease severe stress.

For the sake of clarity, when referring to pain, it can be understood as primary pain or secondary pain. In the context of the present invention, the term “primary pain” or just “pain” is to be understood as pain that is associated with significant emotional distress or functional disability in which no underlying condition adequately accounts for the pain or its impact. Examples of primary pain conditions include fibromyalgia, complex regional pain syndrome, headache, migraine, irritable bowel syndrome, or non-specific low-back pain.

In the context of the present invention, the term “secondary pain” is to be understood as pain that may initially be regarded as a symptom of other diseases having said disease being the underlying cause. Examples of secondary pain are pain related to cancer, pain related to blisters, surgery, injury, internal disease, disease in the muscles, bones or joints, headaches or nerve damage. Primary pain and secondary pain can coexist.

As used herein and unless otherwise specified, the term “pain” is to be understood as a distressing feeling or an unpleasant sensory and emotional experience associated with, or resembling that associated with, actual or potential tissue damage. Pain can be regarded as physiological responses of the human body to e.g. tissue injury and infection. Two response phases can be distinguished: acute and chronic. The acute phase being an early, non-specific phase which is characterized by local vasodilatation and an increased capillary permeability, an accumulation of fluid and proteins in the interstitial spaces, the migration of neutrophils away from the capillaries and the release of inflammatory mediators such as cytokines. The release of said pro-inflammatory mediators leads among others to a perception by the human body which is defined as “pain”.

Chronic pathological pain endures beyond the resolution of the pain source and can even deeply impact the quality of life of people suffering therefrom.

In some embodiments, the pain may be neuropathic pain. Such neuropathic pain is to be understood as pain due to peripheral or central nervous system injury, wherein the sensitization of pain is generally evoked by sensory stimuli in the absence of noxious stimuli.

In some embodiments, primary pain is selected from the list comprising: headaches, migraine, physiological pain, or physical ailments.

As used herein, and unless otherwise specified, the term “headache” is to be understood as the symptom of pain in the face, head, or neck. It can occur as a tension-type headache (normal headache that cause pain in the head, face, or neck), cluster headache, sinus headache, or migraine. Cluster headaches are severely painful headaches that occur on one side of the head and come in clusters i.e. cycles of headache attacks, followed by headache-free periods. Sinus headaches co-occur with sinus infection symptoms like fever, stuffy nose, cough, congestion, and facial pressure. Migraine is a headache that is intense and severe and often have other symptoms in addition to head pain such as nausea, pain behind one eye or ear, pain in the temples, seeing spots or flashing lights, sensitivity to light and/or sound, temporary vision loss, vomiting.

Pain associated with cancer is one of the most severe forms of pain. Such pain can be further exacerbated by cancer treatments, including radiation therapy and chemotherapy. The present formulation may be used to treat cancer associated pain in muscles, bones, and joints. The present formulation can also be used in combination with currently available treatments for such pain to provide an enhanced and/or additive relief effect.

In a next embodiment, the present invention provides a combination as defined herein for use in the treatment of a disorder selected from the list comprising: pain, neurodegenerative disorders, post-traumatic stress syndrome, or blisters is disclosed, wherein said use includes the alleviation of secondary symptoms caused by said disorders.

In some embodiments, a long-term treatment may be necessary for the alleviation of said secondary symptoms. Some of the disorders which can be treated with the combination require a long-term treatment in order to sufficiently alleviate the secondary symptoms associated with said disorders.

In some embodiments, this might involve a lifelong treatment. In other embodiments, this may be a treatment up until complete disappearance of the secondary symptoms caused by the disorders.

In some embodiments, said secondary symptoms may be chronic or recurring symptoms.

In a further embodiment, the present invention provides a combination as defined herein for use in the treatment of a disorder selected from the list comprising: pain, neurodegenerative disorders, post-traumatic stress syndrome, or blisters is disclosed, wherein said use includes the alleviation of secondary symptoms caused by said disorders such as selected from the list comprising: secondary pain, itch, secondary impetigos, swelling, inflammation or bacterial infection.

In some embodiments, said secondary pain is selected from the list comprising: pain related to blisters, cancer, surgery, injury, internal disease, disease in the muscles, bones or joints, or nerve damage; in particular muscle sprains, muscle aches, bruises, arthritis, pain associated with cancer, joint pain such as shoulder, knee, elbow, back pain; surgical pain, preoperative and postoperative pain.

As used herein and unless otherwise specified, the term “anti-inflammatory property” is to be understood as a property appointed to for example substances/compounds/treatment/drugs which reduce inflammation or swelling.

As used herein and unless otherwise specified, the term “analgesic property” is to be understood as a property appointed to for example substances/compounds/treatment/drugs which reduces pain, for example by interacting in various ways on peripheral and central nervous systems.

As used herein and unless otherwise specified, the term “antipruritic property” is to be understood as a property appointed to for example substances/compounds/treatment/drugs which inhibit itch (also referred to as: pruritis).

As used herein and unless otherwise specified, the term “antifungal properties” is to be understood as a property appointed to for example substances/compounds/treatment/drugs which prevent or treat various fungal conditions.

As used herein and unless otherwise specified, the term “antibacterial properties” is to be understood as a property appointed to for example substances/compounds/treatment/drugs which suppress bacterial growth or their ability to reproduce or completely eliminate bacteria.

The analgesic effects of THC are linked to the agonism of cannabinoid receptors CB₁ and CB₂. Other effects comprise muscle relaxation and antiemesis. CBD, on the other hand shows activity including: 5-HT_{1A} receptor agonism, GPR55 antagonism, negative allosteric modulation of CB₁, TRPV1 activation, PPARγ activation and reuptake inhibition. CBD also appears to show activity at both CB₁ and CB₂ receptors while indirectly activating the endogenous cannabinoid signalling. All of this is believed to effectuate the anxiolytic, neuroprotective, anti-inflammatory and immunomodulatory properties of CBD.

Also, research has shown similar activity of CBD against gram-positive bacteria (e.g. Staphylococcus aureaus and Streptococcus pneumoniae) compared to conventional antibiotics (e.g. vancomycin), further explaining the role of CBD in bacterial infections.

Furthermore, the specific combination of THC and CBD used in combination therapies is shown to have synergistical properties and, for example, suppress neuroinflammation and reduce muscle spasticity. Also, CBD or THC alone as well as combinations are shown to have anti-inflammatory and anti-hyperalgesia effects. Furthermore, research shows that either CBD alone or CBD in combination with THC may alter fear memory and may have positive effects on anxiety in the case of post-traumatic stress syndrome.

The combination of THC and CBD has been shown to be efficient in the treatment of neuropathic pain, for example allodynia. In this case, CBD has shown to enhance the pain-relieving actions of THC.

As used herein and unless otherwise specified, the term “allodynia” is to be understood as a central pain sensitization following from stimulations which are normally not painful. Often, these stimulations are repetitive.

In some embodiments, the pain may be neuropathic pain.

In some embodiments, the combination may have at least one property selected from the list comprising: anti-inflammatory, analgesic, antipruritic, antifungal or antibacterial agent properties.

In another embodiment, the present invention provides a combination as defined herein for use in the treatment of a disorder selected from the list comprising: pain, blisters, neurodegenerative disorders or post-traumatic stress syndrome is disclosed, wherein said use includes the reduction of opioid consumption/dependency in the treatment of said disorders.

The current combination provides for a suitable alternative for medicines containing for example opioid substances. As these opioid substances are shown to entail a substantial risk of addiction and may result in fatal overdoses, the current combination offers an alternative fulfilling a long-term need, more specifically in this area of treatment. The current combination reduces the risk of drug addiction (e.g. opioid addiction) especially when long-term treatments are necessary. The longer the duration of drug use such as morphine, oxycodone or other strong analgesics, the higher the risk of addiction. Also, the longer these analgesics are used, the higher the doses because of substantive amount of habituation. This may contribute to the risk of a fatal overdose. Therefore, the use of the current combination provides for a suitable alternative having a positive effect of reducing the risk of drug addiction compared to the use of conventional drugs (e.g. opioids).

Finally, the anti-hyperalgesia effects as shown for the combination of THC and CBD may offer a valuable treatment for opioid-induced hyperalgesia which is associated with long-term use of opioids such as morphine, oxycodone and methadone.

In another embodiment, the combination for use as defined herein may comprise one or more additional pharmaceutically active agents suitable for use in the treatment of said disorders.

In some embodiments, the additional pharmaceutically active agents may act as ligands of the bodily cannabinoid receptors (e.g. cannabinoid receptor type 1 (CB₁), cannabinoid receptor type 2 (CB₂)) and other cannabinoid receptors.

In some embodiments, the uptake of the combination may happen via the pulmonary route.

In other embodiments, the additional pharmaceutically active agents may be drug classes which contribute to the reduction of secondary symptoms such as pain and inflammation.

### EXAMPLES

The following disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

### Example 1: Pulmonary Formulation for Use in Dry Powder Inhalers

In this example the formulation is processed via electrospraying to produce drug nanoparticles.

TABLE 1
Composition of a 1% wt/wt Δ⁹-THC (10 mg/mL)
pulmonary formulation for use in dry powder inhalers.

| | Unit | % wt/wt |
|---|---|---|
| THC | % | 1 |
| Ethanol | % | 38 |
| Propylene Glycol | % | 11 |
| Water (buffered) | % | 45 |
| Kolliphor RH40 | % | 0.5 |
| Butylated Hydroxy anisole (BHA) | % | 0.01 |
| Sucralose | % | 5 |

Formulations were processed by electrospraying using a Fluidnatek® LE10 lab line from Bioinicia S.L. (Valencia, Spain) with a variable high-voltage 0-30 kV power supply. Formulations were fed into a 5-mL syringe and pumped for each solution through a stainless-steel needle injector. Samples were collected on a grounded metallic flat plate. The applied voltage, flow-rate, and tip-to-collector distance were optimized based on visual observation of the Taylor cone formation and no droplet deposition on the collector. Different biopolymers from different origins were evaluated as potentials matrices such as Whey protein concentrate (WPC) with a protein content of 80%, polysaccharide maltodextrin, vegetal protein zein or plastic derived polymer polyvinylpyrrolidone (PVP). Surfactant was added to all solutions at a concentration of 6 wt. % with respect to the polymer weight in order to improve its sprayability according to previous authors.

### Example 2: Pulmonary Formulation for Use in Soft Mist Inhalation Devices

Each single dose (may also be referred to as: “puff”) (volume of 15 microliter) of this formulation from the soft mist inhalation device would deliver an effective dose of 150 μg to the subject in need thereof.

TABLE 2
Composition of a 1% wt/wt Δ⁹-THC (10 mg/mL) pulmonary
formulation for use in soft mist inhalation devices.

| | Unit | % wt/wt |
|---|---|---|
| THC | % | 1 |
| Ethanol | % | 38 |
| Propylene Glycol | % | 11 |
| Water (buffered) | % | 45 |
| Kolliphor RH40 | % | 0.5 |
| Butylated Hydroxy anisole (BHA) | % | 0.01 |
| Sucralose | % | 5 |

### Example 3: Spray Drying of CBD for Inhalation Particles for Pulmonary Delivery

Equipment Set-Up

Spray drying was performed using a spray dryer equipped with a small-sized cyclone (a ProCepT spray dryer). A bi-fluid nozzle with an orifice of 0.4 mm was used to atomize the liquid feed. The nozzle air rate was adapted in order to obtain a particle size close to the desired range (for example between 1 and 5 μm). Following parameters are as an example that could be used during spray drying: inlet airflow (0.30 m³/min), cyclone gas (100 L/min) inlet temperature (120° C.), and spray rate (4 g/min).

Preparation of Liquid Feed

A liquid feed was prepared by mixing a CBD solution in a solvent such as ethanol with another excipient (for example mannitol, maltodextrin, trehalose, lactose, (2-Hydroxypropyl)-beta-cyclodextrin (HPBCD), random methyl-beta-cyclodextrin) in water. A surfactant such as lecithin may be added to the suspension which leads to a more stable system with less sedimentation after 48 hours storage.

The ethanol/water ratio was 1/2 (v/v) but altering the solvent ratio from 1/2 to 1.5/1 (v/v) or 1/1 (v/v) water/ethanol may lead to a more stable (i.e. no phase separation, no precipitation) liquid feed for the formulation with 1/12 (w/w) CBD/excipient.

Assessment of Different Formulation Excipients

Keeping the CBD/excipient ratio constant at 1/12 (w/w), obtained high yields after spray drying the suspensions containing cyclodextrins as excipient (i.e. 62% and 76% for excipients HPBCD and random methyl-BCD respectively), compared with other excipients such as lactose, mannitol, maltodextrin and trehalose 51%) (Table 3).

The addition of a surface-active amino acid (i.e. leucine) representing 10% (w/w) of the combined weight of CBD and excipient lead to a significant increase in yield in the case of maltodextrin, trehalose or lactose (Table 3). The same increase in yield was observed after the addition of leucine representing 5% (w/w) of the combined weight of CBD and other excipients in the case of lactose, HPBCD, and RMBCD (Table 3). In particular, a formulation containing 5% or 10% (w/w) of leucine increased the yield to respectively 73% and 74% compared with to a formulation containing 0% of leucine (yield 47%). The positive effect of leucine on the yield does not necessarily increase the yield further for the cyclodextrin based formulations (Table 3). Leucine typically migrates to the surface of the droplet during the drying phase in the spray dryer resulting in an increased concentration of leucine at the outer crust which lowers the cohesiveness of the particle. Leucine has the capability of increasing the flowability of inhalation powders.

TABLE 3
Yield, mean particle size and size fraction (0-5 μm).

| | | Leucine | Yield | D50 | % |
|---|---|---|---|---|---|
| Sample | Excipient | (%)) | (%) | (μm) | (0 μm < x < 5 μm) |
| 1 | Mannitol | 0 | 28 | 3.80 | 62 |
| 2 | Maltodextrin | 0 | 51 | 2.02 | 91 |
| 3 | Trehalose | 0 | 40 | 1.63 | 94 |
| 4 | Lactose | 0 | 47 | 1.54 | 97 |
| 5 | HPBCD | 0 | 62 | 1.93 | 94 |
| 6 | Random | 0 | 76 | 1.91 | 95 |
| | methyl-BCD | | | | |
| 7 | Maltodextrin | 10 | 60 | 3.38 | 69 |
| 8 | Trehalose | 10 | 66 | 2.08 | 92 |
| 9 | Lactose | 10 | 74 | 2.10 | 91 |
| 10 | Lactose | 5 | 73 | 2.04 | 91 |
| 11 | HPBCD | 5 | 62 | 1.89 | 95 |
| 12 | Random | 5 | 73 | 2.06 | 93 |
| | methyl-BCD | | | | |

Scanning Electron Microscopy

Scanning electron microscopy (SEM) pictures show that spherical particles can be obtained from the powders with trehalose and lactose as excipient (data not shown). It was confirmed by the SEM pictures that leucine migrated to the outer crust during the drying phase, as particles with a rougher surface were obtained when leucine was added to the formulation, compared to the smooth particles without leucine (data not shown).

More wrinkled particles (which provide for better inhalation properties) were obtained when HPBCD and random methyl-BCD were used as excipient due to the smaller diffusion rate of these large molecules (i.e. cyclodextrins) (SEM picture not shown). The surfaces of the particles with and without leucine were similar confirming that leucine did not migrate to the outside of the droplets. This observation supported the assumption made before that leucine was captured in the cyclodextrins. A fraction of the obtained particles seemed to have broken during the drying process as broken shells were visible on the SEM pictures.

Modulated Differential Scanning Calorimetry

Pure CBD and a selection of SD samples were analyzed via modulated differential scanning calorimetry (mDSC) to evaluate the solid state of CBD in the spray dry powder. For example, no melting peak of CBD (i.e. 67° C., FIG. 1A) was present on the mDSC graphs of the formulations with lactose or random methyl-BCD as excipients (i.e. without leucine) (FIG. 1B).

Optimization of Process with RMBCD as Excipient

A formulation with random methyl beta-cyclodextrin (RMBCD) and no leucine might give best yield out of all assessed formulations (i.e. 76%) but might contain an amount of broken particles in the powder which might affect the inhalation properties of the product.

Furthermore, although a formulation with a 2/1 (v/v) water/ethanol ratio with a RMBCD excipient of 5% (w/w) and an inlet temperature of 120° C. had a 76% yield, the preparation resulted in a sedimentation/phase-separation only 5 minutes after preparation and thus is a less stable liquid feed (Table 5).

Altering the solvent ratio from 2/1 to 1/1 (v/v) water/ethanol may lead to a more stable (i.e. no phase separation, no precipitation) liquid feed for the formulation with 1/12 (w/w) CBD/RMBCD which remained clear. However, as an increase in ethanol might lead to a higher abundance of broken particles (Table 5, sample 2), the solvent ratio can have a different solvent ratio to avoid the broken particles while still maintaining a stable solution (e.g. 1.5/1 (v/v) water/ethanol).

Lowering the solid content might lead to a slower crust formation which might avoid the breaking of particles. The solid content might be lowered from 5% (w/w) to 2.5% (w/w) but does not necessarily lead to a better yield (Table 5, sample 3).

Lowering the inlet temperature (for example from 120° C. to 100° C.) may result in a lower amount of broken particles in the spray dried powder, exemplified by the high yield (71%) and high liquid feed stability (Table 5, sample 4).

TABLE 5
Yield and liquid feed stability of a CBD
formulation with RMBCD as excipient.

| | | | H₂O/ | | | | | % |
|---|---|---|---|---|---|---|---|---|
| | | Solid | EtOH | | | Liquid | | (0 μm < |
| | | content | ratio | IT | Yield | feed | D50 | x < 5 |
| Sample | Excipient | (w/w) | (v/v) * | (° C.)# | (%) | stability | (μm) | μm) |
| 13 | RMBCD | 5% | 2/1 | 120 | 76% | + | 1.91 | 95.42 |
| 14 | RMBCD | 5% | 1/1 | 120 | 67% | ++ | 2.12 | 89.35 |
| 15 | RMBCD | 2.5% | 1/1 | 120 | 55% | ++ | 2.32 | 82.36 |
| 16 | RMBCD | 5% | 1/1 | 100 | 71% | ++ | 2.55 | 81.79 |

* Solvent ratio
# Inlet Temperature

Conclusion

In summary, a liquid feed comprising a CBD solution, ethanol, water, and a excipient solution (e.g. mannitol, maltodextrin, trehalose, lactose, (2-Hydroxypropyl)-beta-cyclodextrin (HPBCD), random methyl-beta-cyclodextrin (RMBCD)) may be suitable to prepare a spray dry formulation for inhalation applications.

In particular, a formulation comprising lactose and 5% (w/w) leucine (surface active compound) with a CBD/lactose ratio of 1/12 (w/w) and a formulation comprising RMBCD with a CBD/RMBCD ratio of 1/12 (w/w) and no leucine have a high yield and stability in the end of the spray drying process. More specifically, a formulation containing random methyl-BCD might have a superior stability compared to the suspension with lactose and does not require leucine for a high yield.

### Example 4: Spray Drying of THC or CBD/THC for Inhalation Particles for Pulmonary Delivery

Equipment Set-Up

Spray drying is performed using a spray dryer which was equipped with a small-sized cyclone (a ProCepT spray dryer). A bi-fluid nozzle with an orifice of 0.4 mm was used to atomize the liquid feed. The nozzle air rate was adapted in order to obtain a particle size close to the desired range (for example between 1 and 5 μm). Following parameters are as an example that could be used during spray drying: inlet airflow (0.30 m³/min), cyclone gas (100 L/min) inlet temperature (100° C. or 120° C.), and spray rate (4 g/min).

Preparation of Liquid Feed

A liquid feed was prepared by mixing a THC or CBD/THC solution in ethanol with another excipient such as lactose or random methyl-beta-cyclodextrin (RMBCD) in water (THC or CBD/THC—excipient ratio of 1/12 (w/w)).

THC Formulation with Lactose as Excipient

A formulation with THC and lactose without leucine (Table 6) at a THC/lactose ratio of 1/12 (w/w), an ethanol/water ratio of 1/2 (v/v), and inlet temperature of 120° C. may lead to a very low yield (i.e. 5%) compared with a CBD formulation comprising lactose and 0% leucine (47%). Addition of 5% leucine may increase yield for the THC formulation to 76%, similar to a CBD formulation comprising lactose and 5% Leucine (see Example 3 and Table 6; i.e. 73%). A similar particle size can be obtained as well.

A formulation wherein the active compound comprises CBD and THC in a 1/1 (w/w) ratio, together with 5% leucine may have a similar yield (74%) and particle size (Table 6). Scanning electron microscopy analysis of the three powders containing leucine may confirm an identical morphology (data not shown).

TABLE 6
Comparison of the CBD and THC formulations comprising lactose.

| | | Leucine | Yield | D50 | % |
|---|---|---|---|---|---|
| Sample | Active compound | (%)) | (%) | (μm) | (0 μm < x < 5 μm) |
| 17 | CBD | 0 | 47 | 1.54 | 97.16 |
| 18 | THC | 0 | 5 | 1.44 | 87.94 |
| 19 | CBD | 5 | 73 | 2.04 | 90.73 |
| 20 | THC | 5 | 76 | 2.23 | 87.89 |
| 21 | CBD/THC | 5 | 74 | 2.16 | 92.82 |
| | (1/1 w/w) | | | | |

THC Formulation with RMBCD as Excipient

A formulation with THC and RMBCD without leucine (Table 6) at a THC/RMBCD ratio of 1/12 (w/w), an ethanol/water ratio of 1/1 (v/v), and inlet temperature of 120° C. remained stable for over 24 hours without mixing indicating that this formulation is certainly suitable for the spray drying.

However, the formulation lead to a slightly lower yield (i.e. 65%) compared with a CBD formulation comprising RMBCD (71%), most likely caused by the slightly lower mean particle size of the product (i.e. 1.99 μm instead of 2.55 μm). (Table 7).

A formulation wherein the active compound comprises CBD and THC in a 1/1 (w/w) ratio, may have a similar yield (64%) and particle size (Table 7), compared to similar formulations containing only CBD or THC. Scanning electron microscopy analysis of the three powders may confirm an identical morphology, although the powder comprising THC may be more cohesive compared to the formulation with CBD, exemplified by larger agglomerates formation (data not shown).

TABLE 7
Comparison of the CBD and THC formulations with RMBCD.

| | Active | Yield | D50 | % |
|---|---|---|---|---|
| Sample | compound | (%) | (μm) | (0 μm < x < 5 μm) |
| 22 | CBD | 71 | 2.55 | 81.79 |
| 23 | THC | 65 | 1.99 | 94.42 |
| 24 | CBD | 64 | 1.96 | 91.59 |

Conclusion

A formulation with lactose and leucine, an active compound CBD, THC, or a combination CBD/THC may have no effect on the different parameters of the formulation as a similar yield and acceptable flowability may be obtained for all powders.

A formulation with RMBCD, an active compound THC, or a combination CBD/THC may have slightly lower yield and increased cohesiveness/stickiness compared to a formulation with RMBCD wherein the active compound is CBD.

### SUMMARY

To retrieve a CBD, THC- or CBD/THC-based formulation for inhalation applications, lactose (an inhalation approved carrier), might be the most interesting excipient as a high yield (i.e. about 73%) could be obtained by adding 5% leucine to the formulation. Alternatively, promising results were obtained with random-methyl-BCD (i.e. cyclodextrin) as excipient, even without the addition of leucine.

1. A combination comprising Tetrahydrocannabinol (THC) and Cannabidiol (CBD) for use in the treatment of a disorder selected from the list comprising: pain, neurodegenerative disorders, post-traumatic stress syndrome, or blisters; characterized in that said combination is formulated in a formulation for pulmonary delivery comprising at least one excipient being a saccharide.

2. The combination for use according to claim 1, wherein said formulation for pulmonary delivery is selected from the list comprising: a liquid formulation, a suspension, a powder or an aerosol.

3. The combination for use according claim 1, wherein the concentration of said excipient is about 5 to 10% (w/w).

4. The combination for use according to claim 1, wherein said excipient has a CBD/excipient, THC/excipient or CBD-THC/excipient ratio of about 1/1 to about 1/20 (w/w), preferably about 1/12 (w/w).

5. The combination for use according to claim 1, wherein said formulation is a powder formulation.

6. The combination for use according to claim 5, wherein said powder formulation comprises particles with an average particle diameter of about and between 0.1 to 100 μm, preferably about and between 0.1 and 10 μm, more preferably about and between 0.1 and 5 μm.

7. The combination for use according to claim 5, wherein said powder formulation comprises particles produced with electrospraying.

8. The combination for use according to claim 2, wherein said aerosol is a propellant-free aerosol.

9. The combination for use according anyone of claims 1 to 8, wherein said formulation is formulated for administration by inhalation.

10. The combination for use according to anyone of claims 1 to 9, wherein said formulation is administered to a subject by means of an inhalation device.

11. The combination for use according to claim 1, wherein said pain is selected from the list comprising: headaches, migraine, physiological pain, or physical ailments.

12. The combination for use according to claim 1, wherein said blisters are caused by burns or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

13. The combination for use according to claim 1, wherein said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

14. The combination for use according to anyone of claims 1 to 13, wherein said use includes the alleviation of secondary symptoms caused by said disorders.

15. The combination for use according to claim 14, wherein said secondary symptoms are selected from the list comprising, secondary pain, itch, secondary impetigos, swelling, inflammation or bacterial infection.

16. A combination comprising Tetrahydrocannabinol (THC) and Cannabidiol (CBD) for use in the treatment of a disorder selected from the list comprising: pain, neurodegenerative disorders, post-traumatic stress syndrome, or blisters; characterized in that said combination is formulated in a formulation for pulmonary delivery, wherein said use includes the reduction of opioid consumption/dependency in the treatment of at least one of said disorders.

17. The combination for use according to anyone of claims 1 to 16, further comprising one or more additional pharmaceutically active agents suitable for use in the treatment of said disorders.
